(19) United States
(12) Patent Application Publication
(10) Pub. No.: US 2007/0012105 A1
(43) Pub. Date: Jan. 18, 2007

(54) Method and apparatus for resistive characteristic assessment

(75) Inventors: Jack R. Engsberg, Eureka, MO (US); David P. Engsberg, New Berlin, WI (US); Sandy A. Ross, Fenton, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Barnes-Jewish Hospital

(21) Appl. No.: 11/180,163

(22) Filed: Jul. 13, 2005

(51) Int. Cl. A61B 5/22

(52) U.S. Cl. 73/379.01

(57) ABSTRACT

A simulated limb for training an evaluator to assess a resistive characteristic of an animal. The limb includes a first member simulating a first portion of the animal, and a second member simulating a second portion of the animal rotatably connected to the first member. The limb also includes a resistance element having a variable resistance that varies an amount of torque required to rotate the second member relative to the first member. A method for training an evaluator to assess a resistive characteristic using a simulated limb includes adjusting the resistance of the resistance element over a range of rotation thereby providing a resistance profile for the simulated limb mimicking the resistive characteristic of the animal. A method for simulating a resistive characteristic of an animal using a simulated limb includes applying a resistance profile to the simulated limb mimicking spasticity or muscle strength over a range of rotation.

This invention was made with government support under grants from the National Institutes of Health (R43 HD044269). The U.S. government has certain rights in this invention.

### BACKGROUND OF THE INVENTION

The present invention relates generally to a method for training healthcare professionals, and more particularly to a method for training an evaluator to assess a resistive characteristic such as spasticity or strength of an animal, particularly a human.

Spasticity is a major issue in cerebral palsy, stroke, spinal cord injury and other neurological disorders. The term spasticity is often used to refer to impairments including muscle hypertonia, hyperactive deep tendon reflex, clonus and velocity dependent resistance to passive stretch. Many surgical and therapeutic procedures are performed and pharmacological drugs used on persons having spasticity to minimize or eliminate impairment. Methods to quantify the various types of spasticity include the Modified Ashworth Scale, electromyography (EMG), deep tendon reflex tests and resistance to passive motion tests. The Modified Ashworth Scale is the standard clinical method for assessing spasticity. In order to use the Modified Ashworth Scale, the evaluator manually moves a patient's passive limb through a range of motion and assesses the resistive characteristics of the limb during the motion. Even with training, the repeatability and reliability of the test has been questioned.

Muscle weakness, or lack of strength, is also a common problem in cerebral palsy, stroke, spinal cord injury and other disabilities. Its existence is a concern when clinicians are considering procedures to improve function. Weakness is related to the inability of the patient to actively produce or control torque about a joint. The standard subjective clinical assessment for evaluating strength is based upon a scale with 11 levels. The first six levels require the patient move or attempt to move the joint in a gravity eliminated position without external resistance. The final five levels require the evaluator to apply a force to the joint and the patient to isometrically resist the applied force. The evaluator senses and then grades the patient's resistance force. The reliability of the strength measure has been a subject of some debate.

A major reason for the lack of reliability in both the Modified Ashworth Scale and manual muscle tests is the different physical characteristics of the evaluators and the patients. Evaluators of differing gender, size and strength may not sense patient resistant forces equally. Similarly, patient spasticity and strength can vary according to time of day, anxiety level, motivation, fatigue, age, gender, size, and strength.

Although reliable and accurate methods exist for assessing spasticity and strength, the methods are complex and require special training and equipment. Despite the lack of reliability and accuracy, the Modified Ashworth Scale and manual muscle test remain the most widely used assessment methods for spasticity and strength due to their simplicity. However, it is believed that reliability and accuracy of the tests can be improved if evaluators could calibrate themselves to a benchmark standard that provides accurate and consistent predefined resistance profiles.

### SUMMARY OF THE INVENTION

Briefly, the present invention includes a simulated limb for training an evaluator to assess a resistive characteristic such as spasticity or strength of an animal. The limb comprises a first member simulating a first portion of the animal and a second member simulating a second portion of the animal rotatably connected to the first member. The limb also has a resistance element operatively connected between the first member and the second member having a selectively variable resistance that varies an amount of torque required to rotate the second member of the simulated limb relative to the first member. In addition, the limb includes a processor operatively connected to the resistance element and adapted for adjusting the resistance of the resistance element over a range of rotation of the second member relative to the first member thereby providing a resistance profile for the simulated limb mimicking the resistive characteristic of the animal.

In another aspect, the invention includes a method for training an evaluator to assess a resistive characteristic such as spasticity or strength of an animal using a simulated limb having a first member, a second member rotatably connected to the first member, and a resistance element operatively connected between the first member and the second member having a selectively variable resistance that varies an amount of torque required to rotate the second member of the simulated limb relative to the first member. The method comprises adjusting the resistance of the resistance element over a range of rotation of the second member relative to the first member thereby providing a resistance profile for the simulated limb mimicking the resistive characteristic of the animal.

In yet another aspect, the invention includes a method for simulating a resistive characteristic such as spasticity or strength of an animal using a simulated limb having a first member, a second member rotatably connected to the first member, and a resistance element operatively connected between the first member and the second member having a selectively variable resistance that varies an amount of torque required to rotate the second member of the simulated limb relative to the first member. The method comprises applying a resistance profile to the simulated limb that mimics at least one of the spasticity and the muscle strength over a range of rotation of the second member relative to the first member.

Other features of the present invention will be in part apparent and in part pointed out hereinafter.

### BRIEF DESCRIPTION OF THE DRAWINGS

FIG. 1 is a perspective of a simulated limb of the present invention;

FIG. 2 is a detail perspective of a simulated limb of the present invention in a first orientation; and

FIG. 3 is a detail perspective of the simulated limb as shown in FIG. 2 in a second orientation.

Corresponding reference characters indicate corresponding parts throughout the several views of the drawings.

### DETAILED DESCRIPTION OF THE PREFERRED EMBODIMENT

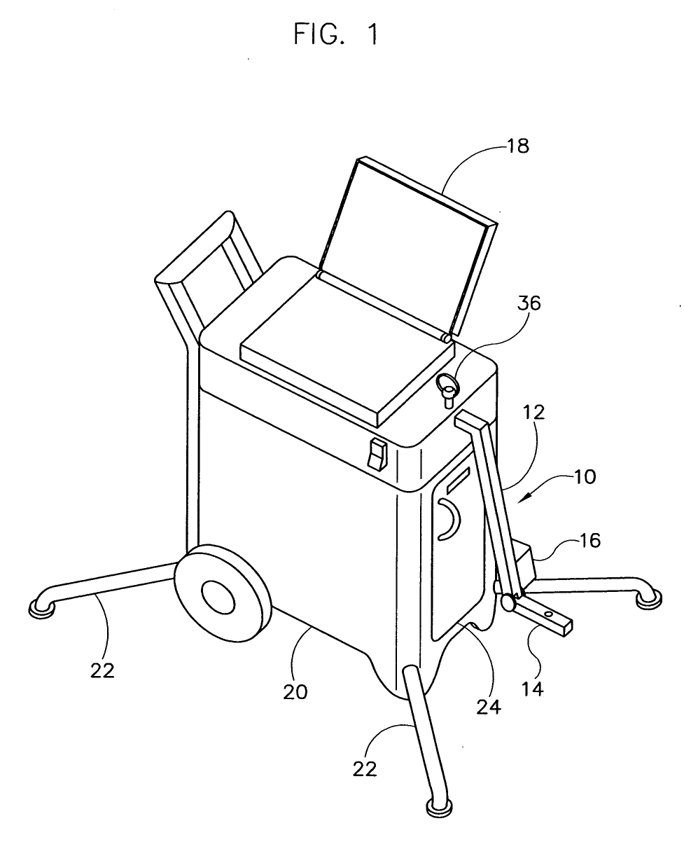

Referring now to the drawings and in particular to FIG. 1, a simulated limb system for training an evaluator to assess a resistive characteristic such as spasticity or strength of an animal such as a human is designated in its entirety by the reference numeral 10. The limb 10 generally comprises a first member (generally designated by 12) simulating a first portion of the animal (e.g., a calf of a human) and a second member (generally designated by 14) simulating a second portion of the animal (e.g., a foot of the human) rotatably connected to the first member. A resistance element 16 is operatively connected between the first member 12 and the second member 14. The element 16 has a selectively variable resistance that varies an amount of torque required to rotate the second member 14 of the simulated limb 10 relative to the first member 12. Although other resistance elements may be used without departing from the scope of the present invention, in one embodiment the resistance element is a DC motor such as a Model SM3450 combination DC motor and servo controller with a peak torque capability of about 5.3 Nm available from Animatics Corporation of Santa Clara, Calif. A conventional power supply (not shown) powers the motor. Although other power supplies may be used without departing from the scope of the present invention, in one embodiment the power supply is a Model PS42V20A power supply available from Animatics Corporation.

A processor 18 is operatively connected to the resistance element 16 and adapted for adjusting the resistance of the resistance element over a range of rotation of the second member 14 relative to the first member 12. By adjusting the resistance of the resistance element 16 over a range of rotation, the processor 18 provides a resistance profile for the simulated limb 10 mimicking the resistive characteristic of the animal. Although other processors 18 may be used without departing from the scope of the present invention, in one embodiment the processor is a conventional PC based laptop computer loaded with Animatics SmartMotor Interface Software. The processor may also include a servo and/or a microprocessor as will be described in further detail below.

In one embodiment, the first and second elements 12, 14, respectively, and the processor 18 are removably mounted on a wheeled cart 20 for transporting the limb 10 to various locations for use. The cart 20 includes outrigger legs 22 for stabilizing the limb 10 and cart during use. Thus, the cart 20 forms a base upon which the limb 10 is mounted. Although the first and second elements 12, 14 may be made of other materials without departing from the scope of the present invention, in one embodiment, these elements are made of square steel tubing. The cart 20 includes a square socket for receiving the first element 12 when connecting the limb 10 to the cart. The cart 20 includes internal spaces for storing alternative limbs and supplies. An access door or panel 24 may be provided to allow access to the interior spaces.

A modular power section (not shown) can be included in the cart 20. In one embodiment, the power section includes a power supply capable of accommodating various standard AC power levels (120V/240V, 50 Hz/60 Hz) with isolation to meet all applicable requirements for medical equipment. In an alternate embodiment, the power section includes a battery power supply. In both embodiments, the power supply includes a main on/off power switch, circuit protection, and a status indicator. A wiring harness may be included to provide communication between various devices on the cart.

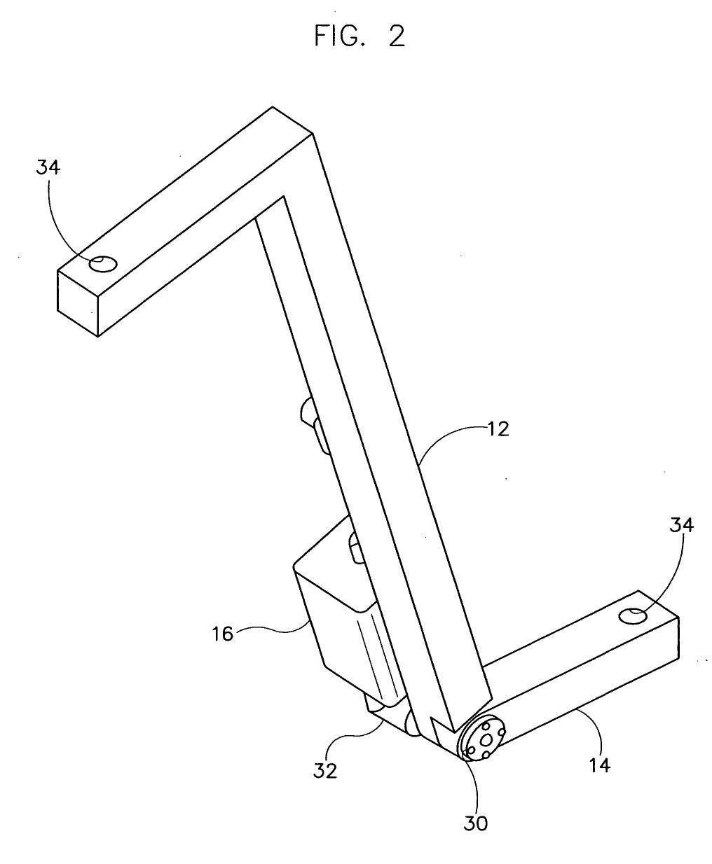

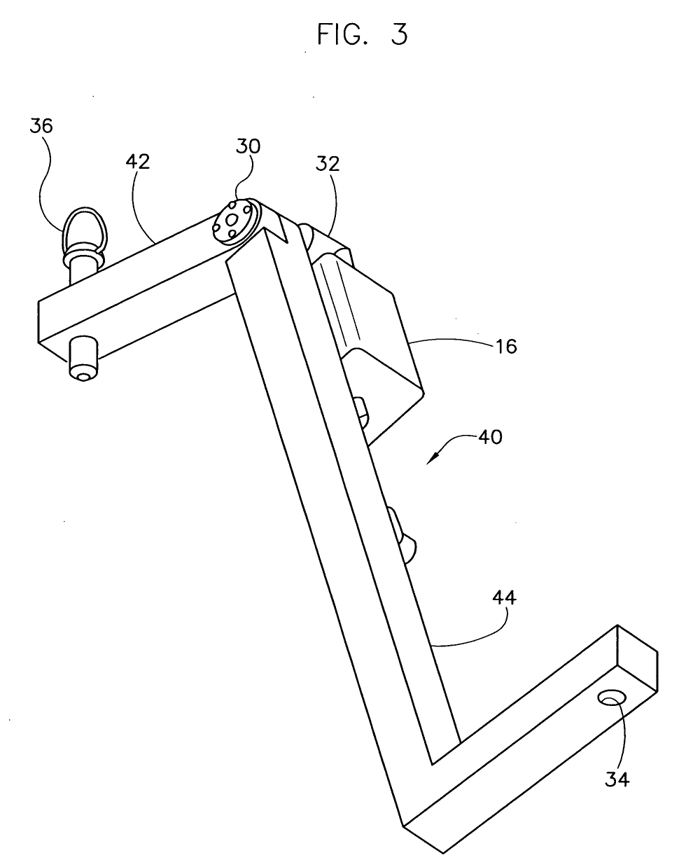

As illustrated in FIG. 2, a bearing 30 connects the first and second elements 12, 14, respectively. A gear mechanism 32 is attached to the motor for increasing the maximum torque (e.g., to approximately 80 Nm). The gear mechanism 32 also changes the direction of the torque so the motor output shaft can be mounted parallel to the second element 14 and at right angles to the bushing 30. A hole 34 is located adjacent the free ends of the first and second elements 12, 14 for receiving a pin 36 (FIG. 3) when connecting the elements to the cart 20. FIG. 2 illustrates the first and second elements 12, 14 in an orientation in which the first element simulates a calf of a human, the second element simulates a foot of the human, and the bearing 30 represents the ankle of the human.

FIG. 3 illustrates a second embodiment of a limb, generally designated by the reference number 40, having a first element 42 and a second element 44. This embodiment has an identical configuration to the embodiment shown in FIG. 2, but is inverted so the element which was previously moveable (i.e., the second element 14) is stationary and thus is referred to as the first element 42 in this orientation and the element which as previously stationary (i.e., the first element 12) is moveable and thus is referred to as the second element 44 in this orientation. In the FIG. 3 orientation, the first element 42 simulates a thigh of a human, the second element 44 simulates a calf of the human and the bearing 30 represents a knee of the human. As will be appreciated by those skilled in the art, limbs having other configurations (not shown) may be developed to simulate other joints in an animal (e.g., an elbow portion of an ape). As will also be appreciated, in some embodiments the first element may not be stationary and/or the second element may not move.

Although the motors used in the previously mentioned embodiments may have other configurations without departing from the scope of the present invention, in one embodiment the motors have integrated servo controllers, gearboxes, and auxiliary input/output devices. The software to control the motor may be any conventional language thereby providing sophisticated control and interface possibilities. Although the resistance element may be made using mechanisms (e.g., comprising weights, springs, conventional dampers, rheological fluid dampers and AC motors), using resistance element comprising a DC motor has several advantages including enabling the device to operate in both a passive and active device, enabling the use of gears to increase available torque, allowing control elements to be computer controlled at high speeds, and potential for battery operation.

The motor embodiment of the resistance element 16 simulates a resistance profile for a joint by rotating its output shaft at varying speeds including stopping and/or rotating backwards. As will be appreciated by those skilled in the art, when a constant current is sent to each of the windings of a motor, a static magnetic field generated within the motor becomes static like a permanent magnet. Because the rotor is attracted to the oppositely charged magnetic pole, the rotor speed can be precisely controlled and evened stopped. To increase the resistance of the joint, more current is delivered to the windings.

Providing the motor 16 with a servo (not shown) allows closed-loop control of position, velocity and/or torque as directed by a program or external controller. In a torque controlled embodiment, the servo controller takes a position, torque and/or velocity command and calculates the torque needed to satisfy that command. Further, the servo outputs to the motor controller a current proportional to desired torque. A high torque setting causes the motor to mimic a stiff joint. Passive resistance simulation can be accomplished by issuing a torque command resulting in a stiffness for the joint proportional to the command.

Many servos have built-in rotational position sensors, allowing the servo to be position controlled, in which case the servo determines the position of the shaft and when the motor shaft reaches the desired position, the servo causes motion to stop. For example, the control may issue a position command to the motor to move the knee joint from a flexed position to an extended position. Simultaneously, the control may issue a torque command (e.g., 30 Nm) to the motor so it only has a pre-selected amount of energy available to make the motion. An evaluator pushing down on the limb would be able to exceed the 30 Nm torque and make the motor move in the reverse direction even as the closed loop position control is driving the leg toward the extending position with as constant torque. As will be appreciated by those skilled in the art, the servo may also include a velocity sensor for sensing the velocity of the second member 14 relative to the first member 12 and controlling movement of the second member based at least in part on the velocity of the second member.

The power and communication links with the motor 16 using standard connectors and wires (not shown). The wires can be attached to the first and second elements 12, 14 and covered with prosthetic foam (not shown) so the limb resembles the physiological feature of the animal being mimicked.

In one embodiment, a joint microcontroller (JMC) (not shown) is operatively connected between the computer 18 and the motor 16. The computer may be used to create the resistance profile, and that profile can be sent and stored on the JMC. It is envisioned that the JMC will control the DC motor through specialized software and that a serial communications system may be used to send information back and forth between the JMC and the motor program. It is envisioned that the DC Motor and a servo controller may be able to replace the JMC.

PC software is used to create resistance profiles for spasticity and strength, and to store profiles and templates for revision and manipulation. Existing software can be used to create resistance profiles mimicked spasticity. The spasticity torque features include: 1) constant resistance, 2) linear resistance, 3) parabolic resistance, 4) resistive spikes, and 5) resistance profiles derived from experimental data. This software can be modified to produce resistance profiles duplicating muscle function (i.e., concentric, eccentric, isometric contractions). Concentric contraction software will produce basically the same resistance profile requirements that the spasticity resistance profiles have except that they will be actively moving the joint through a range of motion instead of resisting through a range of motion. The four key resistance features (constant, linear, parabolic, experimental) will form the base profiles. For example, a constant resistance profile of 50 Nm would indicate that the evaluator would sense a constant torque of 50 Nm over the entire range of motion. For a linear resistance profile, the evaluator would sense a torque of 0 Nm at the start of the range of motion and an 80 Nm torque at the end of the range of motion. A parabolic resistance profile would indicate that the evaluator would sense a parabolic resistance characteristic of a typical muscle length-tension curve. The parabolic profile has no resistance (i.e., 0 Nm torque) at the beginning and end of the range of motion and a maximum of 80 Nm at the middle of the range of motion. The basic parabola is the same as the spasticity parabola (x=−(16y+120)12) except that y is within the range of from 5 to −5 instead of from 0 to −10. It is envisioned that the software can include special features such as a spike and/or fatigue. The spike would be used to create a rebound effect. The fatigue feature would primarily be used with the isometric contractions where there would be a decline in resistance over time. Eccentric contractions will cause the joint to tend to maintain in a fixed position (e.g., full extension). The position will be changed by the evaluator as he or she moves the joint to the end range of motion (e.g., full knee flexion). Isometric contraction software will produce active resistance profiles that maintain a constant torque at a fixed joint angle. Thus, unlike the other active and passive tests where the joint moves through a range of motion, an isometric contraction would involve no change in joint angle. As will be appreciated by those skilled in the art, the changes in torque provide a resistance profile that is variable over the range of rotation of the element and/or over a predetermined period of time. In this way, the resistance profile of the resistance element may be an active propulsive torque or a passive resistive torque.

In one embodiment, at least a portion of the resistance profile mimics at least a portion of the Modified Ashworth scale for assessing spasticity. In an alternate embodiment, at least a portion of the resistance profile mimics at least a portion of the manual muscle test assessment strategy.

The limb 10 described above may be used to train an evaluator to assess a resistive characteristic such as spasticity or strength of an animal. The resistance of the resistance element 16 is adjusted over a range of rotation of the second member 14 relative to the first member 12 to provide a resistance profile for the simulated limb mimicking the resistive characteristic of the animal. The evaluator is instructed to apply a force to the second member 14 of the simulated limb 10 so the evaluator can experience a simulation of the resistive characteristic of the animal. Using this method, the evaluator can practice evaluating the resistive characteristics of the patient to improve the accuracy of the evaluation.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense.

What is claimed is:

1. A method for training an evaluator to assess a resistive characteristic such as spasticity or strength of an animal using a simulated limb having a first member, a second member rotatably connected to the first member, and a resistance element operatively connected between the first member and the second member having a selectively variable resistance that varies an amount of torque required to rotate said second member of the simulated limb relative to said first member, said method comprising adjusting the resistance of the resistance element over a range of rotation of the second member relative to the first member thereby providing a resistance profile for the simulated limb mimicking the resistive characteristic of the animal.

2. A method in accordance with claim 1 further comprising instructing the evaluator to apply a force to the second member of the simulated limb so the evaluator can experience a simulation of the resistive characteristic of the animal.

3. A method in accordance with claim 1 wherein the resistance profile of the simulated limb comprises an active propulsive torque.

4. A method in accordance with claim 1 wherein the resistance profile of the simulated limb comprises a passive resistive torque.

5. A method in accordance with claim 1 wherein at least a portion of the resistance profile of the simulated limb mimics at least a portion of the Modified Ashworth scale for assessing spasticity.

6. A method in accordance with claim 1 wherein at least a portion of the resistance profile of the simulated limb mimics at least a portion of a manual muscle test assessment strategy.

7. A method in accordance with claim 1 wherein the resistance profile of the simulated limb is variable over time.

8. A method in accordance with claim 1 wherein the resistance profile of the simulated limb comprises a constant torque.

9. A method for simulating a resistive characteristic such as spasticity or strength of an animal using a simulated limb having a first member, a second member rotatably connected to the first member, and a resistance element operatively connected between the first member and the second member having a selectively variable resistance that varies an amount of torque required to rotate said second member of the simulated limb relative to said first member, said method comprising applying a resistance profile to the simulated limb that mimics at least one of the spasticity and the muscle strength over a range of rotation of the second member relative to the first member.

10. A method in accordance with claim 9 wherein at least a portion of the resistance profile mimics at least a portion of the Modified Ashworth scale for assessing spasticity.

11. A simulated limb for training an evaluator to assess a resistive characteristic such as spasticity or strength of an animal, said limb comprising:
a first member simulating a first portion of the animal;
a second member simulating a second portion of the animal rotatably connected to the first member;
a resistance element operatively connected between the first member and the second member having a selectively variable resistance that varies an amount of torque required to rotate said second member of the simulated limb relative to said first member; and
a processor operatively connected to the resistance element and adapted for adjusting the resistance of the resistance element over a range of rotation of the second member relative to the first member thereby providing a resistance profile for the simulated limb mimicking the resistive characteristic of the animal.

12. A simulated limb in accordance with claim 11 wherein at least a portion of the resistance profile mimics at least a portion of at least one of the Modified Ashworth scale for assessing spasticity and the manual muscle test assessment strategy.

13. A simulated joint in accordance with claim 11 further comprising at least one of a position sensor and a velocity sensor operatively connected to said second member for sensing at least one of a position and a velocity of the second member relative to said first member, said at least one position sensor and velocity sensor being operatively connected to the processor for relaying said sensed position or velocity to the processor.

14. A simulated limb in accordance with claim 11 wherein the resistance element comprises an electric motor.

15. A simulated limb in accordance with claim 11 wherein the resistance profile is variable over at least one of the range of rotation and a predetermined period of time.

16. A simulated limb in accordance with claim 11 wherein the resistance profile comprises a constant torque.

17. A simulated limb in accordance with claim 11 wherein the resistance profile of the resistance element comprises an active propulsive torque.

18. A simulated limb in accordance with claim 11 wherein the resistance profile of the resistance element comprises a passive resistive torque.
